(19) United States
(12) Patent Application Publication
(10) Pub. No.: US 2026/0284301 A1
(43) Pub. Date: Sep. 24, 2026

(54) Drug Delivery Device With Skin Contact Sensor

(71) Applicant: Becton, Dickinson and Company, Franklin Lakes, NJ (US)

(72) Inventors: Steve Beguin, Nenzlingen (CH); Stuart Plascott, Foxrock (IE); Christian Sandmann, Wayne, NJ (US); Natasha G. Bolick, Raleigh, NC (US); Wendy D. Woodley, Cary, NC (US); Sylvine Raverdy, River Edge, NJ (US)

(21) Appl. No.: 18/997,111

(22) PCT Filed: Sep. 8, 2023
(86) PCT No.: PCT/US23/32242

§ 371 Date: Jan. 20, 2025

Related U.S. Application Data
(60) Provisional application No. 63/405,099, filed on Sep. 9, 2022.

(51) Int. Cl. A61M 5/142

(52) U.S. Cl. CPC A61M 5/14248; A61M 2005/14252; A61M 2205/13; A61M 2205/18; A61M 2205/3306; A61M 2205/3375; A61M 2205/3576

(57) ABSTRACT

Provided herein is a wearable drug delivery device including a housing having a skin-facing surface defining a cannula opening, the skin-facing surface configured to releasably attach to a patient's skin, a microcontroller received within the housing and including at least one processor, a sensor in communication with the microcontroller, the sensor configured to detect the patient's skin, a container disposed within the housing and configured to store a pharmaceutical composition therein, and a cannula in fluid communication with the container, the cannula transitionable from an initial position in which the cannula is disposed within the housing to a use position in which the cannula extends through the cannula opening.

### CROSS-REFERENCE TO RELATED APPLICATION

The present application claims priority to U.S. Provisional Application No. 63/405,099, entitled “Drug Delivery Device with Skin Contact Sensor” filed Sep. 9, 2022, the entire disclosure of which is hereby incorporated by reference in its entirety.

### BACKGROUND OF THE INVENTION

### Field of the Invention

The present disclosure relates to wearable drug delivery devices, and, in particular, wearable drug delivery devices capable of detecting separation of the device from a user's skin.

### Description of Related Art

Medical devices, such as wearable injectors, autoinjectors, and other drug delivery devices, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period. After the device delivers the pharmaceutical composition to the patient, the patient may subsequently remove and dispose of the device.

In certain circumstances, the drug delivery device must be worn for a period of time extending several minutes, hours, or days prior to drug delivery. In other circumstances, injections are administered during a longer period of time, or with multiple sequential injections (e.g. insulin, Alzheimer's medications, Parkinson's medications, chemotherapies, etc.). In these situations, a soft catheter may be used. However, device detachment (partial, complete, transient, and/or permanent) may lead to migration of the catheter outside the target injection site and cause failure. Moreover, this failure has the potential to be silent and may go unnoticed by the patient. Accordingly, there is a need in the art for robust sensors and alerts for use with wearable drug delivery devices.

### SUMMARY OF THE INVENTION

Provided herein is a wearable drug delivery device including a housing having a skin-facing surface defining a cannula opening, the skin-facing surface configured to releasably attach to a patient's skin, a microcontroller received within the housing and including at least one processor, a sensor in communication with the microcontroller, the sensor configured to detect the patient's skin, a container disposed within the housing and configured to store a pharmaceutical composition therein, and a cannula in fluid communication with the container, the cannula transitionable from an initial position in which the cannula is disposed within the housing to a use position in which the cannula extends through the cannula opening.

In one embodiment, the sensor is a proximity sensor.

In one embodiment, the drug delivery device is secureable to a patient.

In one embodiment, the drug delivery device is a wearable device.

In one embodiment, the proximity sensor is a capacitive sensor, an inductive sensor, and/or an ultrasonic sensor.

In one embodiment, the sensor is an ultrasonic sensor and comprises an emitter and a receiver.

In one embodiment, the sensor is an optical sensor.

In one embodiment, the optical sensor includes an emitter and a receiver.

In one embodiment, the drug delivery device further includes an adhesive patch arranged between the housing and the patient's skin.

In one embodiment, the sensor is at least partially arranged in and/or on a surface of the adhesive patch.

In one embodiment, the sensor includes a severable electrical circuit, a strain gauge, and/or a piezeoelectric material.

In one embodiment, the adhesive patch includes one or more openings therethrough, and wherein the sensor is aligned with the one or more openings.

In one embodiment, the sensor is an optical sensor or an ultrasonic sensor.

In one embodiment, the sensor includes an emitter and a receiver.

In one embodiment, the sensor includes a switch.

In one embodiment, the switch is a mechanical switch.

In one embodiment, the processor is programmed or configured to receive data from the sensor, the data relating to whether the drug delivery device is in contact with the patient's skin, and determine, based on a status of the wearable drug delivery device and the data, whether the wearable drug delivery device is properly arranged on the patient's skin.

In one embodiment, the processor is programed or configured to provide a feedback indication to the patient based on at least one of correct placement and orientation of the device.

In one embodiment, the processor is programed or configured to provide instructions to adjust or correct placement of the drug delivery device prior to, during, and/or after an injection.

In one embodiment, the wearable drug delivery device further includes at least one of an indicator capable of communicating a condition of the wearable drug delivery device to a user and a communication module configured to transmit data relating to a status of at least one property of the wearable drug delivery device.

In one embodiment, at least one processor is further programmed or configured to, upon determining that the wearable drug delivery device is not properly arranged on the patient's skin, provide, through the indicator and/or the communication module, an alert.

In one embodiment, the alert is an audible, tactile, and/or visual indication.

In one embodiment, the container is a flexible container.

In one embodiment, the container is a syringe barrel.

In one embodiment, the device further includes a motor configured to expel the pharmaceutical composition from the container.

In one embodiment, the data received from the sensor is used to determine whether the delivery device should avoid completion or pursue delivery if contact with the patient is disrupted before a second condition is met.

In one embodiment, the second condition is at least one of delivery completion, pressure change below a threshold indicative of tissue absorption, and deployment or removal of an insertion mechanism.

### BRIEF DESCRIPTION OF THE DRAWINGS

FIG. 1 is a perspective view of a drug delivery device according to one aspect or embodiment of the present application;

FIG. 2 is a perspective view of the drug delivery device of FIG. 1, with a top cover removed;

FIG. 3 is a schematic of the drug delivery device of FIG. 1;

FIG. 4 is a schematic of the drug delivery device of FIG. 1;

FIG. 5 is a schematic of the drug delivery device of FIG. 1;

FIG. 6 is a schematic of the drug delivery device of FIG. 1;

FIG. 7 is a diagram of a non-limiting embodiment or aspect of an environment in which systems, devices, and/or methods described herein may be implemented; and

FIG. 8 is a diagram of a non-limiting embodiment or aspect of components of one or more devices of FIG. 7.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

As used herein, the term “cannula” may refer to needles, cannulae, catheters, and like structures that allow for fluid communication between a pharmaceutical composition container and a patient.

As used herein, the term patient may refer to a human or non-human animal.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible. Any known electronic communication protocols and/or algorithms can be used such as, for example, TCP/IP (including HTTP and other protocols), WLAN (including 802.11a/b/g/n and other radio frequency-based protocols and methods), analog transmissions, Global System for Mobile Communications (GSM), 3G/4G/LTE, BLUETOOTH, ZigBee, EnOcean, TransferJet, Wireless USB, and the like known to those of skill in the art. In some non-limiting embodiments, a message may refer to a network packet (e.g., a data packet and/or the like) that includes data.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

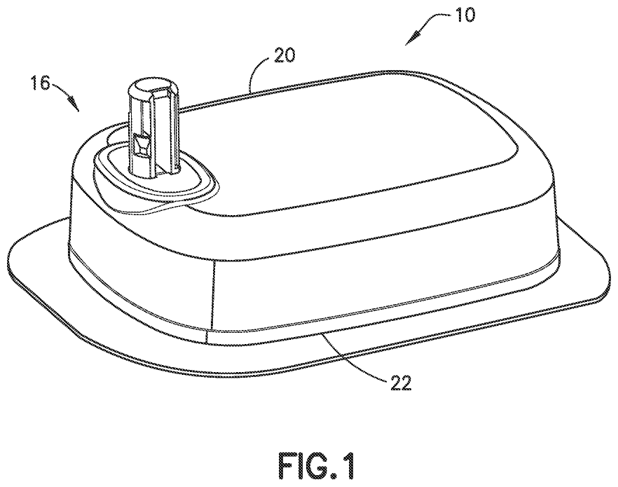

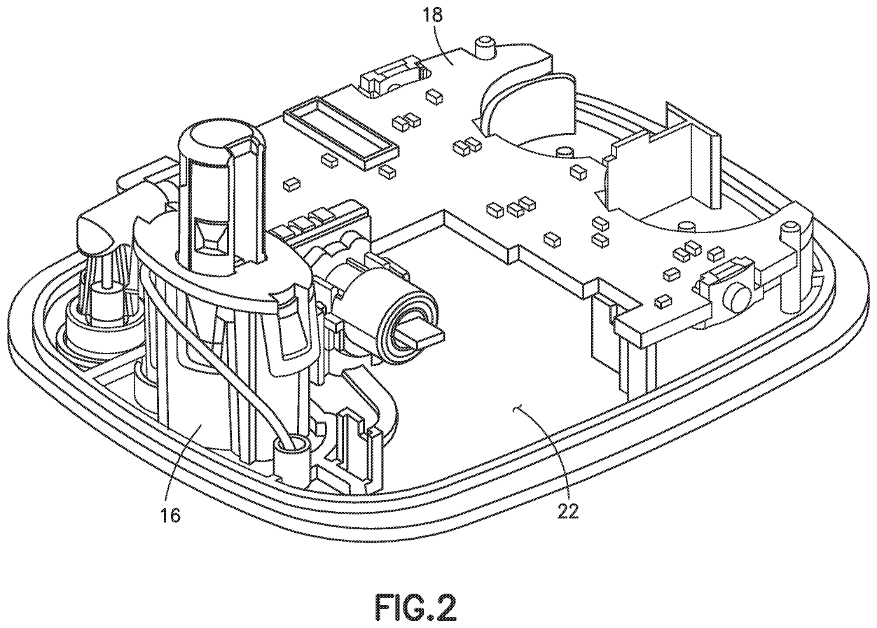

Referring to FIGS. 1 and 2, a drug delivery device 10 includes a reservoir, an insertion mechanism 16, control electronics 18, a cover 20, and a base 22. In non-limiting embodiments, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

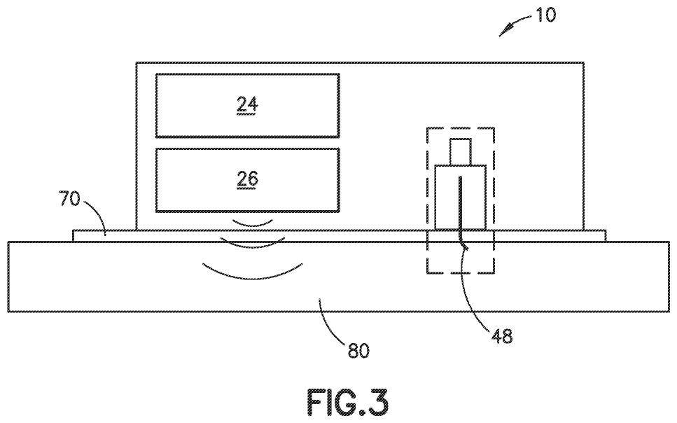

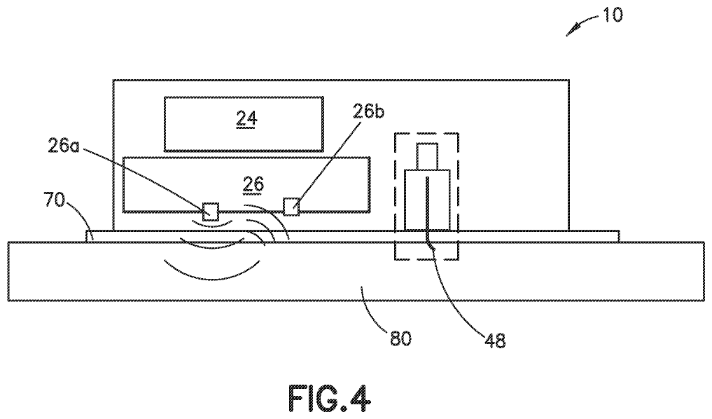

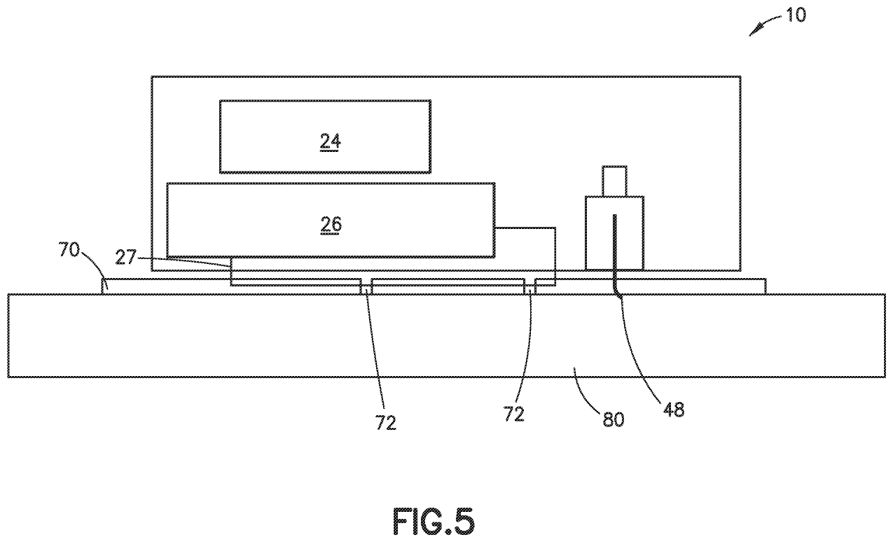

Referring to FIGS. 3-5, drug delivery device 10 may further include an adhesive patch 70 for securing the drug delivery device 10 to the patient's skin 80, and may further include a computing device, e.g. microcontroller 24, and a sensor 26 in communication with the microcontroller. Microcontroller 24 may include one or more components of a computing device as described herein (and shown for example, in FIG. 9), for example one or more processors and, optionally, memory, for example to store programming instructions to provide functionality as described below. FIGS. 3-5 show non-limiting embodiments of sensors that may be useful in detecting whether drug delivery device 10 is in proximity to (e.g., attached to) a patient's skin. While capacitive and/or inductive sensors (FIG. 3), optical and/or ultrasonic sensors (FIG. 4), and sensors including severable electrical circuits, strain gauges, and/or piezoelectric materials (FIG. 5) are exemplified, those of skill will appreciate that other sensors, including switches, such as mechanical switches, may be used to determine proximity to a patient's skin. For example, a mechanical switch may be biased to a first configuration, e.g., by a spring or other resilient member. When drug delivery device 10 is properly applied to patient's skin 80, a switch may be held against the force of the bias in a second configuration. Upon partial and/or complete detachment or dislodgment from skin 80, a switch may transition, partially or fully, to first configuration, which may be detected by microcontroller 24, which may, as described herein, alert a patient to the condition. In non-limiting embodiments, drug delivery device may include any type, number, and arrangement of sensors 26 as described herein.

Turning to FIG. 3, shown is a non-limiting embodiment in which sensor 26 is a proximity sensor, such as a capacitive sensor and/or an inductive sensor, which can detect changes in the environment around the sensor based on change in generated electric and/or magnetic fields due to different materials (e.g., air versus skin) present in the vicinity of the sensor. A change in the detected field may, when processed by microcontroller 24, determine whether drug delivery device 10 has been moved relative to the patient's skin 80, such that cannula 48 may not be at, or capable of reaching, the proper injection depth. Suitable capacitive and inductive sensors are known to those of skill in the art, and are available commercially.

With regard to FIG. 4, shown is a non-limiting embodiment in which sensor 26 is an optical sensor and/or an ultrasonic sensor. In non-limiting embodiments, optical and/or ultrasonic sensor 26 includes an emitter 26a and a detector 26b, which may be within the same sensor unit, or within different sensor units. In non-limiting embodiments, emitter 26a is a light-emitting diode (LED) and detector 26b is a photodiode. In non-limiting embodiments, adhesive patch 70 includes one or more openings therethrough, and sensor 26 (and/or components thereof, such as emitter 26a and/or detector 26b) may be aligned with the one or more openings, such that there is, in some embodiments, no adhesive pad between sensor 26 and patient's skin 80. As above, change in the detected signal (e.g., ultrasound or optical) may, when processed by microcontroller 24, determine whether drug delivery device 10 has been moved relative to the patient's skin 80, such that cannula 48 may not be at, or capable of reaching, the proper injection depth.

With regard to FIG. 5, shown is a non-limiting embodiment in which sensor 26 is incorporated at least partially in adhesive patch 70, for example by way of a severable electric circuit, strain gauge, and/or piezoelectric material. By “incorporated” it is contemplated that sensor may be received at least partially within adhesive patch 70 and/or on a surface of adhesive patch 70. Adhesive patch 70 may, as described above, have one or more openings 72, for example to correspond with a pre-cut or severable portion of patch 70. Such openings 72 may distinguish sections of patch 70 that are heat staked to a base of drug delivery device 10, and those sections that are not. Such sensors may detect a change in orientation of drug delivery device 10 relative to patient's skin 80, for example based on deformation of adhesive patch 70, which may include one or more portions of sensor 26. As above, change in the detected signal (e.g., interruption in electrical circuit, amplitude of strain, and/or piezoelectric signal) may, when processed by microcontroller 24, determine whether drug delivery device 10 has been moved relative to the patient's skin 80, such that cannula 48 may not be at, or capable of reaching, the proper injection depth.

In non-limiting embodiments of the drug delivery device 10 described herein, microcontroller 24, which is in communication with sensor 26, may be programmed or configured to compare a signal received from sensor 26, and compare to an appropriate reference value corresponding to the type of sensor 26 included in drug delivery device 10, to determine whether drug delivery device 10 is properly arranged on patient's skin 80. In non-limiting embodiments, upon determining that drug delivery device 10 is not properly arranged on patient's skin 80, an alert may be transmitted through any visual, audible, and/or tactile means, by virtue of microcontroller 24 being in communication with an indicating device of the appropriate type (e.g., a light source, such as an LED, a speaker, and/or a device that generates a tactile indication). In non-limiting embodiments, microcontroller 24 may also store data relating to the arrangement of drug delivery device 10 in memory, to provide a history for later review and/or comparison.

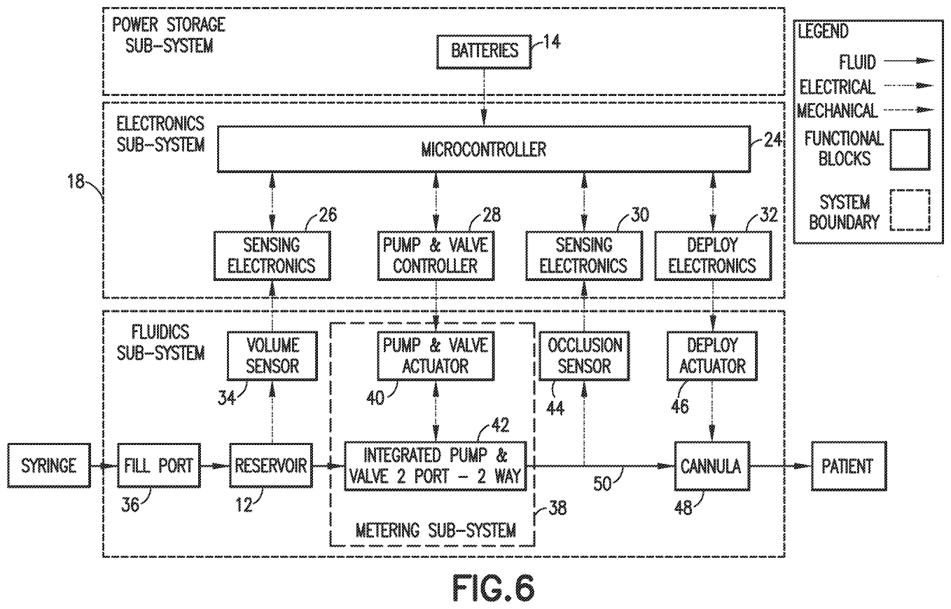

Referring to FIG. 6, shown is a schematic view of possible components of drug delivery device 10. In non-limiting embodiments, drug delivery device 10 may include a power source 14. In non-limiting embodiments, power source 14 is a DC power source including one or more batteries, though those of skill will appreciate that other sources of power can be included. As noted above, drug delivery device 10 may also include control electronics 18, including a computing device (e.g., microcontroller 24), sensor 26, a pump and valve controller 28, sensing electronics 30, and deployment electronics 32, which control the actuation of the drug delivery device 10. The drug delivery device 10 includes a fluidics sub-system that includes the reservoir 12, a volume sensor 34 for the reservoir 12, a reservoir fill port 36, and a metering system 38 including a pump and valve actuator 40 (e.g., a motor) and a pump and valve mechanism 42. Those of skill in the art will appreciate that any suitable container may be used in the context of the present inventions. For example, while FIG. 6 references a syringe having fill port 36 and reservoir 12, other suitable containers, such as flexible containers, may be used with the present invention. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. Drug delivery device 10 may operate in the same manner as discussed in U.S. Pat. No. 10,449,292, incorporated herein by reference in its entirety.

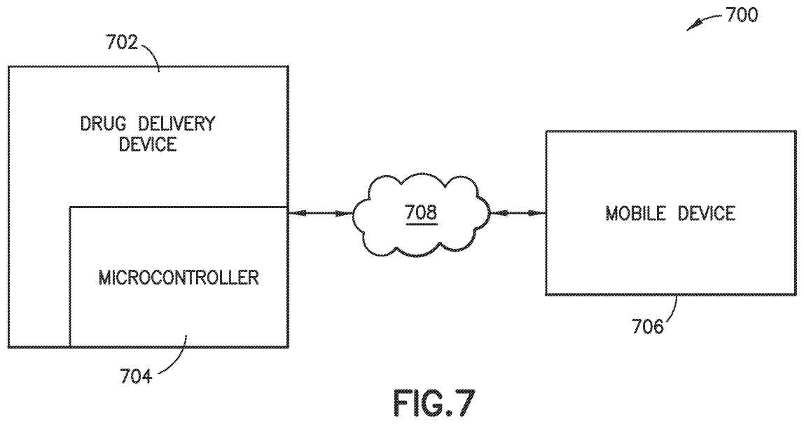

With reference to FIG. 7, shown is a diagram of an example environment 700 in which devices, systems, and/or methods, described herein, may be implemented. The environment 700 may include drug delivery device 702 including a microcontroller 704 and one or more sensors as described herein, a computing device, such as a mobile device 706 associated with a patient, and/or a communication network 708. Drug delivery device 702 and mobile device 706 may interconnect (e.g., establish a connection to communicate) via wired connections, wireless connections, or a combination of wired and wireless connections.

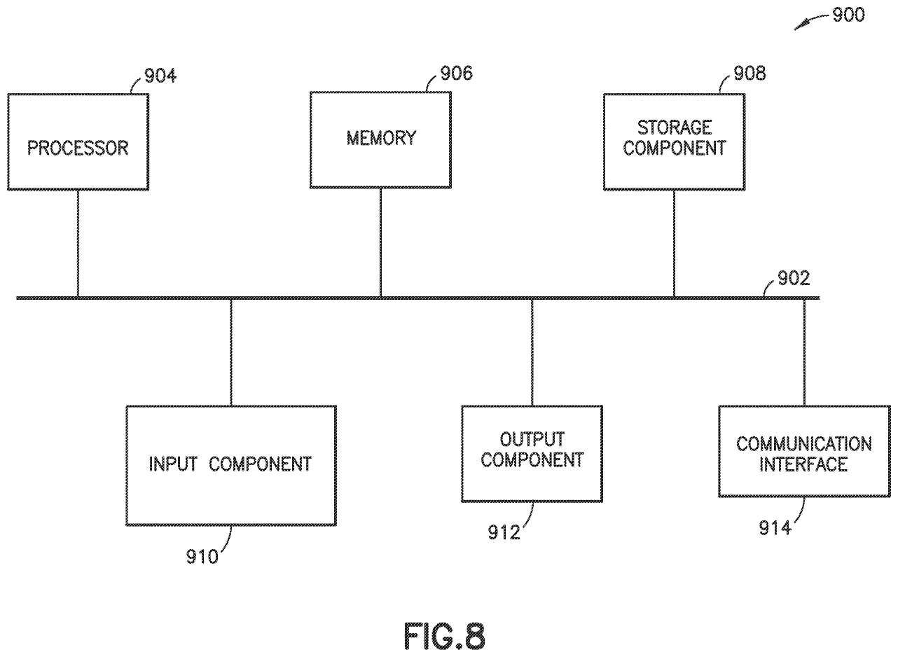

Drug delivery device 702, which can be a wearable drug delivery device as described herein, may include, as described above, one or more sensors in communication with microcontroller 704, which may also include a communication interface to transmit and/or receive data from user device 706. One or both of microcontroller 704 and user device 706 may be a computing device as described herein, including any component described herein and/or exemplified in FIG. 8, described below.

Communication network 708 may include one or more wired and/or wireless networks. For example, communication network 708 may include a BLUETOOTH connection (e.g., between drug delivery device 702 and user device 706), a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a low-power wide area network (LPWAN), an ultra-wideband network (UWB), a metropolitan area network (MAN), a telephone network (e.g., the public switched telephone network (PSTN) and/or the like), a private network, an ad hoc network, an intranet, the Internet, a fiber optic-based network, a cloud computing network, and/or the like, and/or a combination of some or all of these or other types of networks.

User device 706 can be a computing device as described herein, in some non-limiting embodiments, a smartphone, smartwatch, tablet, laptop computer, desktop computer, or other computing device. User device 706 can be programmed or configured to communicate, for example through communication network 708, with other systems, for example healthcare systems associated with a pharmaceutical manufacturer, a device manufacturer, a clinical trial sponsor, and/or a physician, for example through an application, such as a mobile application, executable on user device 706.

With reference to FIG. 8, shown is a diagram of example components of an exemplary computing device useful in the devices, systems, and methods described herein. Such a computing device 900 may correspond to a component within the drug delivery device as described herein and/or a user device, such as a mobile phone, tablet, laptop computer, smart watch or other personal communication device, for example as shown in exemplary environment 700 of FIG. 7. As shown in FIG. 9, a computing device 900 may include bus 902, processor 904, memory 906, storage component 908, input component 910, output component 912, and/or communication interface 914.

Bus 902 may include a component that permits communication among the components of a computing device 900. In some non-limiting embodiments, processor 904 may be implemented in hardware, software, or a combination of hardware and software. For example, processor 904 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 906 may include random access memory (RAM), read-only memory (ROM), and/or another type of dynamic or static storage memory (e.g., flash memory, magnetic memory, optical memory, and/or the like) that stores information and/or instructions for use by processor 904.

Storage component 908 may store information and/or software related to the operation and use of computing device 900. For example, storage component 908 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 910 may include a component that permits computing device 900 to receive information, such as via user input (e.g., a touch screen display, a keyboard, a keypad, a mouse, a button, a switch, a microphone, and/or the like). Additionally or alternatively, input component 910 may include a sensor for sensing information (e.g., a global positioning system (GPS) component, an accelerometer, a gyroscope, an actuator, and/or the like). Output component 912 may include a component that provides output information from a computing device (e.g., a display, a speaker, one or more light-emitting diodes (LEDs), and/or the like).

Communication interface 914 may include a transceiver-like component (e.g., a transceiver, a separate receiver, and transmitter, etc.) that enables device to communicate with other devices, such as via a wired connection, a wireless connection, or a combination of wired and wireless connections. Communication interface 914 may permit computing device 900 to transmit and/or receive information from another device. For example, communication interface 914 may include an Ethernet interface, an optical interface, a coaxial interface, an infrared interface, a radio frequency (RF) interface, a universal serial bus (USB) interface, a Wi-Fi® interface, a cellular network interface, BLUETOOTH interface, and/or the like. In non-limiting embodiments, communication interface 914 operates through one or both of near-field communication and RFID. Suitable communication protocols and methods for securing communications between communication interface 914 and a communication interface of another device, such as a computing device (e.g., desktop computer, laptop computer, smartphone, smart watch, PDA, tablet, etc. ,) can include encryption, e.g., using a secure socket layer (SSL) (e.g., by using public/private key pairs as are known in the art). Additional security protocols are disclosed in, for example, U.S. Pat. Nos. 9,445,264 and 9,463,325, the contents of which are hereby incorporated by reference in their entirety.

A computing device may perform one or more processes described herein. A computing device may perform these processes based on processor 904 executing software instructions stored by a computer-readable medium, such as memory 906 and/or storage component 908, and/or being instructed by a separate computing device. A computer-readable medium (e.g., a non-transitory computer-readable medium) is defined herein as a non-transitory memory device. A non-transitory memory device includes memory space located inside of a single physical storage device or memory space spread across multiple physical storage devices. Software instructions may be read into memory 906 and/or storage component 908 from another computer-readable medium or from another device via communication interface 914. When executed, software instructions stored in memory 906 and/or storage component 908 may cause processor 904 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A drug delivery device comprising:
a housing comprising a skin-facing surface defining a cannula opening, the skin-facing surface configured to releasably attach to a patient's skin;
a microcontroller received within the housing and comprising at least one processor;
a sensor in communication with the microcontroller, the sensor configured to detect the patient's skin;
a container disposed within the housing and configured to store a pharmaceutical composition therein; and
a cannula in fluid communication with the container, the cannula transitionable from an initial position in which the cannula is disposed within the housing to a use position in which the cannula extends through the cannula opening.

2. The drug delivery device of claim 1, wherein the sensor is a proximity sensor.

3. The drug delivery device of claim 1, wherein the drug delivery device is secureable to a patient.

4. The drug delivery device of claim 1, wherein the drug delivery device is a wearable device.

5. The drug delivery device of claim 2, wherein the proximity sensor is a capacitive sensor, an inductive sensor, and/or an ultrasonic sensor.

6. The drug delivery device of claim 5, wherein the sensor is an ultrasonic sensor and comprises an emitter and a receiver.

7. The drug delivery device of claim 1, wherein the sensor is an optical sensor.

8. The drug delivery device of claim 7, wherein the optical sensor comprises an emitter and a receiver.

9. The drug delivery device of claim 1, further comprising an adhesive patch arranged between the housing and the patient's skin.

10. The drug delivery device of claim 9, wherein the sensor is at least partially arranged in and/or on a surface of the adhesive patch.

11. The drug delivery device of claim 10, wherein the sensor comprises a severable electrical circuit, a strain gauge, and/or a piezeoelectric material.

12. The drug delivery device of claim 9, wherein the adhesive patch comprises one or more openings therethrough, and wherein the sensor is aligned with the one or more openings.

13. The drug delivery device of claim 12, wherein the sensor is an optical sensor or an ultrasonic sensor.

14. The drug delivery device of claim 13, wherein the sensor comprises an emitter and a receiver.

15. The drug delivery device of claim 1, wherein the sensor comprises a switch.

16. The drug delivery device of claim 15, wherein the switch is a mechanical switch.

17. The drug delivery device of claim 1, wherein the processor is programmed or configured to:
receive data from the sensor, the data relating to whether the drug delivery device is in contact with the patient's skin; and
determine, based on a status of the wearable drug delivery device and the data, whether the wearable drug delivery device is properly arranged on the patient's skin.

18. The drug delivery device of claim 1, wherein the processor is programed or configured to provide a feedback indication to the patient based on at least one of correct placement and orientation of the device.

19. The drug delivery device of claim 1, wherein the processor is programed or configured to provide instructions to adjust or correct placement of the drug delivery device prior to, during, and/or after an injection.

20. The drug delivery device of claim 17, wherein, the wearable drug delivery device further comprises at least one of an indicator capable of communicating a condition of the wearable drug delivery device to a user and a communication module configured to transmit data relating to a status of at least one property of the wearable drug delivery device.

21.-27. (canceled)
